(19) United States
(12) Patent Application Publication
(10) Pub. No.: US 2025/0181161 A1
(43) Pub. Date: Jun. 5, 2025

(54) BRAIN-COMPUTER TARGET READING METHOD BASED ON DYNAMIC GRAPH REPRESENTATION NETWORK AND SYSTEM THEREOF

(71) Applicant: Hangzhou Dianzi University, Hangzhou City (CN)

(72) Inventors: Wanzeng KONG, Hangzhou City (CN); Jiabin ZHU, Hangzhou City (CN); Haixiao WANG, Hangzhou City (CN)

(21) Appl. No.: 18/921,450

(22) Filed: Oct. 21, 2024

(30) Foreign Application Priority Data
Nov. 30, 2023 (CN) 202311636395.X

(51) Int. Cl. G06F 3/01

(52) U.S. Cl. CPC G06F 3/015

(57) ABSTRACT

A brain-computer target reading method based on a dynamic graph representation network and a system thereof are provided. The system includes a dynamic temporal graph constructing module, a dual-branch graph pooling module and a dynamic temporal attention module. The dynamic temporal graph constructing module captures a time-varying connectivity relationship between Electroencephalography (EEG) signal channels. The dual-branch pooling module retains local structure information and global structure information in the process of purifying features, which reduces the loss of effective information. Finally, the dynamic temporal attention module allows a model to pay more attention to task-related representations, thus improving the overall classification performance of the model. Compared with the existing event-related potential identification method, the result of the brain-computer target reading method is better. The brain-computer target reading method overcomes the limitation of static graph network in terms of dynamically capturing the time-varying connectivity between the EEG signal channels.

### CROSS-REFERENCE TO RELATED APPLICATION

This patent application claims the benefit and priority of Chinese Patent Application No. 202311636395.X filed with the China National Intellectual Property Administration on Nov. 30, 2023, the disclosure of which is incorporated by reference herein in its entirety as part of the present application.

### TECHNICAL FIELD

The present disclosure belongs to the technical field of brain-computer interfaces, and relates to a brain-computer target reading method based on a dynamic graph representation network, in particular to a method of capturing the time-varying connectivity between channels of Electroencephalography (EEG) signals using a dynamic temporal graph constructing module and extracting a task-related feature using a dual-branch graph pooling module and a dynamic temporal attention module, so as to identify event-related potentials.

### BACKGROUND

The Brain-Computer Interface (BCI) technology realizes the direct communication between a human brain and an external device. In a BCI system, the commonly used non-invasive Electroencephalography (EEG) technology is widely used to record the brain activity. Rapid Serial Visual Presentation (RSVP) is a widely used BCI paradigm based on the EEG, which is often used to carry out a target detection task. Under this paradigm, a subject needs to identify a specific target image in a rapidly displayed image sequence. When the subject detects the target image, the EEG signals will induce corresponding Event-Related Potentials (ERP), such as P300, N100, N400, etc. By analyzing these ERP components, researchers can judge whether the subject has successfully observed the target image.

At present, many researches rely on a convolutional neural network or a cyclic neural network to extract features from EEG data. However, these methods usually ignore the connectivity relationships between different channels of EEG signals, which are critical for EEG analysis. In addition, a model based on the convolutional neural network or the cyclic neural network often limits feature extraction to the Euclidean domain. In order to solve these problems, researchers developed a graph neural network, which is a powerful tool for learning non-Euclidean data representation and is especially suitable for analysis of EEG signals.

The existing brain-computer target reading method based on the graph neural network can effectively model the complex interaction between brain areas and the connectivity relationship between electrodes in the EEG data, thus significantly improving the accuracy and efficiency of classifying EEG signals. Therefore, the method according to the present disclosure has attracted more and more attention in the research field. Specifically, these methods construct an adjacency matrix in a graph neural network by analyzing the physical distance between electrodes or the correlation between EEG channel signals. However, such methods based on the static graph neural network fail to fully consider the temporal dynamics of the connectivity between electrodes in EEG signals, thus failing to fully capture the dynamic changes of the brain activity. Such limitation may result in the insufficient performance of the model in the process of adapting to the changes of the brain network between different individuals or the same individual under different conditions, which limits the classification effect and the generalization ability.

### SUMMARY

An object of the present disclosure is to provide a brain-computer target reading method of EEG signals based on a dynamic graph representation network.

In a first aspect, the present disclosure provides a brain-computer target reading method based on dynamic graph features, which specifically includes the following steps:
Step 1: acquiring Electroencephalography (EEG) data to obtain a data set including a plurality of samples;
Step 2: slicing each sample to obtain a plurality of time slices xᵢ;
Step 3: for each sample, constructing a dynamic temporal graph; wherein the dynamic temporal graph includes a constructed sequence graph Gᵢ corresponding to each time slice xᵢ; for each sequence graph Gᵢ, constructing an adjacency matrix Aᵢ using two one-dimensional vectors;
Step 4: extracting time domain features; the time domain feature \tilde{x}ₗ of each time slice xᵢ is extracted by two-dimensional convolution, and zero-padding is performed on the time slice xᵢ, such that dimensions of the time slice xᵢ and the time domain feature \tilde{x}ₗ are same;
Step 5: extracting a graph feature; wherein, the graph feature hᵢ of each sequence graph Gᵢ is extracted using a dynamic graph homogeneous network;
Step 6: purifying the graph feature hᵢ using a dual-branch graph pooling module; wherein the dual-branch graph pooling module performs a graph pooling operation based on local topology information and a graph pooling operation based on global topology information on the graph feature hᵢ respectively, and combines obtained graph features to obtain a pooled feature hᵢ^{pool},
Step 7: extracting a global graph feature gᵢ of each sequence graph based on the pooled feature hᵢ^{pool};
Step 8: extracting a task-related feature from global graph features gᵢ of all sequence graphs to obtain a weighted global graph feature gᵢ^{attn},
Step 9: using an adaptive average pooling function to aggregate the weighted global graph feature gᵢ^{attn}, and classifying a obtained feature to obtain a prediction result y; and judging whether a target object is observed by a subject when collecting an EEG signal according to the prediction result y.

Preferably, in Step 1, the collected EEG data is preprocessed; and the pre-processing operation includes filtering, down-sampling and re-reference.

Preferably, in Step 3, the sequence graph Gᵢ=<Vᵢ, Eᵢ, Aᵢ>; where Vᵢ is nodes in the sequence graph, which represents channels of EEG signals; Eᵢ represents a set of edges for connecting nodes, and Aᵢ represents an adjacency matrix of the sequence graph.

Preferably, an expression of the adjacency matrix Aᵢ is:

$$\begin{matrix}{A}_{i} = tanh ( Φ \cdot {Ψ}^{T} - Ψ \cdot {Φ}^{T} ) \\ idx , idy = arg topk ( {A}_{i} [ : , : ] ) \\ A\_i [ - idx , - idy ] = 0\end{matrix}$$

where tanh(·) is a hyperbolic tangent activation function; Φ and Ψ are two learnable one-dimensional vectors; argtopk(·) is a return function for a maximum element subscript; k is a preset parameter.

Preferably, an expression of extracting a graph feature hᵢ in Step 5 is:

$${h}_{i}^{v} = MLP ( ( 1 + ϵ ) \cdot \tilde{{x}_{l}^{v}} + \tilde{{x}_{l - 1}^{v}} + {\sum }_{u ∈ 𝒩 ( v )} {w}_{ij} \cdot \tilde{{x}_{l}^{u}} )$$

where hᵢ^{v} represents a feature of a node v in a sequence graph Gᵢ after passing through the dynamic graph homogeneous network; MLP(·) is a multi-layer perceptron process; wᵢⱼ is an edge weight obtained from the adjacency matrix Aᵢ, and ∈ is a learnable parameter; \tilde{x}ₗ^{v} and \tilde{x}ₗ₋₁^{v} are time domain features corresponding to a single node in two adjacent time slices, respectively.

Preferably, the Step 6 includes:
6-1, pooling the graph feature based on a local topology;
wherein, a channel topology graph of a sample is divided into five areas according to a frontal lobe, a parietal lobe, an occipital lobe, a left temporal lobe and a right temporal lobe; an importance score of each node in each area is calculated respectively; a plurality of nodes with highest importance in each area are selected to pool, the graph feature hᵢ to obtain a locally pooled graph feature hᵢ^{local_pool};
6-2: pooling the graph feature hᵢ using the method based on the global topology via an expression:

$${h}_{i}^{global \_ pool} = bias ( {N}^{′} ) + {\sum }_{k = 0}^{N - 1} weight ( {N}^{′} , k ) ★ {h}_{i}$$

where * represents an effective two-dimensional cross-correlation operator; N and N″ represent a number of EEG channels before and after global topology pooling, respectively, and hᵢ^{global_pool} represents a sequence graph feature after global topology pooling; and
6-3, performing fusion of results of dual-branch pooling using an expression:

$${h}_{i}^{pool} = Conv 2 d ( {h}_{i}^{global \_ pool} + {h}_{i}^{local \_ pool} )$$

where Conv2d(·) is a two-dimensional convolution operation.

Preferably, in Step 7, the global graph feature gᵢ is extracted by a Softmax aggregation function, with an expression:

$${g}_{i} = {\sum }_{v ∈ {h}_{i}^{pool}} \frac{exp ( β \cdot v )}{{\sum }_{v ∈ {h}_{i}^{pool}} exp ( β \cdot u )} \cdot v$$

wherein v and u are nodes in the pooled feature hᵢ^{pool}, and β is a learnable parameter.

Preferably, Step 8 includes:
calculating a weight scoreᵢ for different time slices using an expression:

$${score}_{i} = σ ( tanh ( \frac{1}{n} {\sum }_{i = 1}^{n} fc ( {g}_{i} ) ) \cdot {g}_{i}^{T} ) ,$$

where fc(·) is a fully connected layer, tanh(·) is a hyperbolic tangent activation function, and σ(·) is a sigmoid activation function; and
after the weight is acquired, weighting and summing the global graph feature gi of each time slice to obtain the weighted global graph feature gᵢ^{attn}.

Preferably, in Step 9, aggregated features are classified by using a linear layer and a Softmax activation function to obtain the prediction result y.

In a second aspect, the present disclosure provides a brain-computer target reading system, including a dynamic temporal graph constructing module, a dual-branch graph pooling module and a dynamic temporal attention module;
wherein the dynamic temporal graph constructing module is configured to divide an EEG sample into a plurality of time slices, construct an adjacency matrix using two learnable vectors for each time slice, and extract a graph feature of each time slice using a dynamic graph homogeneous network;
the dual-branch graph pooling module is configured to perform graph pooling operation based on local topology information and graph pooling operation based on global topology information on the graph feature, respectively, and combine obtained graph features to obtain a pooled feature;
the dynamic temporal attention module is configured to extract a global graph feature of each sequence graph based on the pooled feature, and extract a feature highly related to a task using an attention mechanism to obtain a global graph feature for prediction and classification.

In a third aspect, the present disclosure provides a computer device, including a memory, a processor and a computer program stored in the memory and operable on the processor, wherein the memory stores the computer program; and the processor executes the brain-computer target reading method.

In a fourth aspect, the present disclosure provides a readable storage medium, having a computer program is stored; wherein the computer program, when executed by a processor, is configured to implement the brain-computer target reading method.

The present disclosure has the following beneficial effects.
1. The dynamic temporal graph constructing module in the neural network model based on the dynamic graph features according to the present disclosure can capture the time-varying connectivity relationship between EEG signal channels. The dual-branch graph pooling module purifies features based on local topology and global topology to prevent the loss of structure information. The dynamic temporal attention module allows a model to pay more attention to task-related features. The present disclosure provides an effective supplement for a graph neural network classification framework in the current event-related potential identification field.
2. The present disclosure eliminates the limitation that the static graph network dynamically captures the time-varying connectivity between EEG signal channels. In addition, with the help of the dual-branch graph pooling module and the dynamic temporal attention module, the model can capture features highly related to a task.

### BRIEF DESCRIPTION OF THE DRAWINGS

FIG. 1 is a flow chart of the present disclosure.

FIG. 2 is a schematic diagram of a model framework according to the present disclosure.

### DETAILED DESCRIPTION OF THE EMBODIMENTS

The method of the present disclosure will be described in detail with reference to the attached drawings.

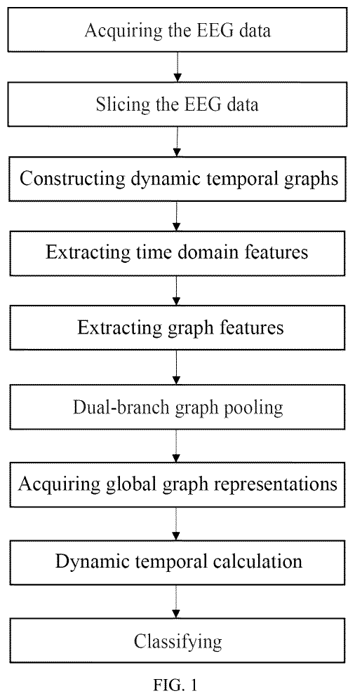

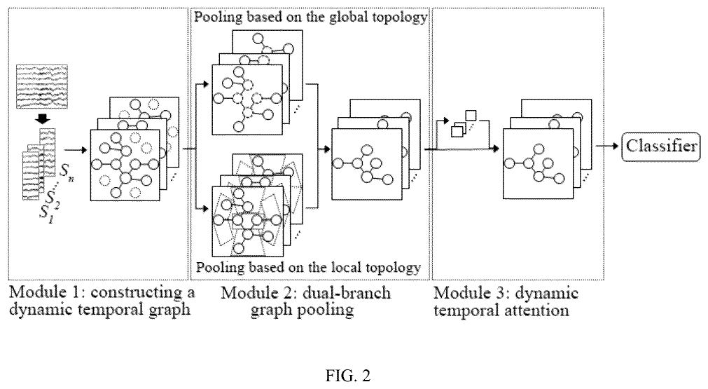

As shown in FIG. 1 and FIG. 2, a brain-computer target reading method based on dynamic graph features is used to judge whether the subject has observed the target object according to the collected EEG data. In this embodiment, the method is specifically used to judge whether a user sees pedestrians in a picture.

The brain-computer target reading method based on the dynamic graph features specifically includes the following Steps 1-9.
Step 1: EEG data is acquired.

The tagged EEG data (namely EEG data) based on the RSVP target detection paradigm is acquired. The EEG data is pre-processed; and the pre-processing operation includes filtering, down-sampling and re-reference to obtain a plurality of EEG samples.
Step 2: time slices are performed on the EEG samples. Each EEG sample x∈R^{N×T} is divided into n time slices xᵢ∈R^{N×(T/n)} evenly, in which i={1, 2, . . . , n}. If T is not divisible by n, zero-padding is performed at the end of the time dimension of the EEG sample x, where N is the number of EEG channels; T is the number of sampling points of a channel.
Step 3: dynamic temporal graphs are constructed. For each time slice xᵢ, the sequence graph Gᵢ=<Vᵢ, Eᵢ, Aᵢ> is constructed, where Vᵢ is a node in the sequence graph, which represents a channel of an EEG signal; Eᵢ represents a set of edges for connecting nodes, and Aᵢ represents an adjacency matrix of the sequence graph.

For each sequence graph Gᵢ, the adjacency matrix Aᵢ is constructed using two learnable one-dimensional vectors, and the construction method is as follows:

$$\begin{matrix}{A}_{i} = tanh ( Φ \cdot {Ψ}^{T} - Ψ \cdot {Φ}^{T} ) \\ idx , idy = arg topk ( {A}_{i} [ : , : ] ) \\ A\_i [ - idx , - idy ] = 0\end{matrix}$$

where tanh(·) is a hyperbolic tangent activation function; Φ and Ψ are two learnable one-dimensional vectors, which are randomly initialized; argtopk(·) is a return function for a maximum element subscript, which is used to return the maximum subscripts of the k elements in the adjacency matrix Aᵢ; the elements of the first k maximum values in the adjacency matrix Aᵢ are retained, and the remaining values are set to zero, thus realizing the sparseness of the matrix; k is a preset parameter, whose value is 10 in this embodiment.
Step 4: a time domain feature is extracted. The time domain feature \tilde{x}ₗ of each time slice xᵢ is extracted by a two-dimensional convolution layer. The convolution kernel size of the two-dimensional convolution is (1, kern_size) and the step size is 1. The time slice xᵢ is zero-padded, so that the feature dimensions of the time slice xᵢ and the time domain feature \tilde{x}ₗ are the same.
Step 5: a graph feature is extracted. In order to transfer and aggregate messages between two adjacent sequence graphs, the graph feature hᵢ of each sequence graph Gᵢ is extracted using a dynamic graph homogeneous network:

$${h}_{i}^{v} = MLP ( ( 1 + ϵ ) \cdot \tilde{{x}_{l}^{v}} + \tilde{{x}_{l - 1}^{v}} + {\sum }_{u ∈ 𝒩 ( v )} {w}_{ij} \cdot \tilde{{x}_{l}^{u}} )$$

where hᵢ^{v} represents a feature of a node v in the sequence graph Gᵢ after passing through the dynamic graph homogeneous network; MLP(·) is a multi-layer perceptron process; wᵢⱼ is an edge weight obtained from the adjacency matrix Aᵢ, and ∈ is a learnable parameter; \tilde{x}ₗ^{v} is a time domain feature corresponding to a node v in the time slice xᵢ, and \tilde{x}ₗ₋₁^{v} is a time domain feature corresponding to a node v in the time slice xᵢ₋₁. N(v) is the set of neighboring nodes of node v; and u is a node in this set.
Step 6: the graph feature hᵢ is purified and the training parameters are reduced using a dual-branch graph pooling module. In view of the local and global topology information included in the EEG signals, a dual-branch graph pooling strategy is used. The graph pooling operation based on local topology information and the graph pooling operation based on global topology information are performed on the graph feature hᵢ respectively, and the obtained graph features are combined to further refine the graph feature hᵢ, which is specifically as follows:

$${h}_{i}^{pool} = Conv 2 d ( {h}_{i}^{global \_ pool} + {h}_{i}^{local \_ pool} )$$

where hᵢ^{local_pool} is the pooled graph feature based on the local topology graph; hᵢ^{global_pool} is the pooled feature based on the global topology graph; hᵢ^{pool} is the feature after passing through the dual-branch pool module.
Step 6-1: the graph feature is pooled using the method based on the local topology. A pooling branch based on the local topology is used to capture the local structure information of the graph. Specifically, the present disclosure divides a channel topology graph of the EEG sample into five areas according to a frontal lobe, a parietal lobe, an occipital lobe, a left temporal lobe and a right temporal lobe. The importance of the node in each area is evaluated by examining the degree of the node. The greater the degree of the node, the more significant its importance in the whole area. The importance score of each node in each area is calculated as follows:

$${score}_{i}^{v} = sum ( {A}_{i} [ v , : ] )$$

where scoreᵢ^{v} represents the degree of the node v in the i-th sequence graph, that is, the number of nodes adjacent to the node v.

The most important k nodes are selected in each area, the features are pooled in this way, and the adjacency matrix is modified accordingly. The specific operation is as follows:

$$\begin{matrix}idx = arg top k ( {score}_{i} ) \\ {h}_{i}^{local\_pool} = {h}_{i} [ idx , : ]\end{matrix}$$

where the function argtopk(·) returns the first k nodes with the largest degrees in each area, hᵢ^{local_pool} is the graph feature after local pooling. k is a preset parameter with a value of 24.
Step 6-2: the graph feature hᵢ is pooled using the method based on the global topology. For pooling based on global topology, this embodiment uses a two-dimensional convolution layer that integrates graph pooling and time domain feature processing to capture the global structure information of the graph feature hᵢ. Specifically, the nodes of the sequence graph are regarded as the number of feature graphs in the convolution layer, and the size of the convolution kernel is the same as that of the convolution kernel of the time domain convolution in Step 4. The feature after the global pooling operation is represented as follows:

$${h}_{i}^{global\_pool} = bias ( {N}^{′} ) + {\sum }_{k = 0}^{N - 1} weight ( {N}^{′} , k ) ★ {h}_{i}$$

where * represents an effective two-dimensional cross-correlation operator; N and N″ represent the number of EEG channels before and after global topology pooling, respectively, and hᵢ^{global_pool} represents a sequence graph feature after global topology pooling. bias is learnable bias term; weight is learnable weight.
Step 6-3: fusion of the results of dual-branch pooling is performed. The feature after dual-branch pooling operation and the adjacency matrix are fused to obtain the final pooling result, respectively. The specific process is as follows.

First, the feature hᵢ^{local_pool} obtained by local pooling is added with the feature hᵢ^{global_pool} obtained by global pooling. Thereafter, a two-dimensional convolution layer with a convolution kernel size of 1×1 is applied to fuse the features to obtain the final pooled feature hᵢ^{pool}. The expression is as follows:

$${h}_{i}^{pool} = Conv 2 d ( {h}_{i}^{global\_pool} + {h}_{i}^{local\_pool} )$$

Step 7: a global graph feature gᵢ of each sequence graph is extracted using the Softmax aggregation function, which is specifically as follows:

$${g}_{i} = {\sum }_{v ∈ {h}_{i}^{pool}} \frac{exp ( β \cdot v )}{{\sum }_{u ∈ {h}_{i}^{pool}} exp ( β \cdot u )} \cdot v$$

where v and u are nodes in the graph feature hᵢ^{pool}, and β is a learnable parameter. exp is natural exponential function.
Step 8: a task-related feature is extracted. Through the dynamic temporal attention module, the task-related feature is acquired. In view of different contributions of each time slice feature to the task, the model needs to pay more attention to the time slice feature related to the task in order to obtain a better classification result. The present disclosure uses an attention mechanism to assign a weight to the global graph feature of each time slice to indicate the importance of each time slice to the task, which are specifically expressed as follows:

$${score}_{i} = σ ( tan h ( \frac{1}{n} {\sum }_{i = 1}^{n} fc ( {g}_{i} ) \cdot {g}_{i}^{T} )$$

where scoreᵢ is the weight of each time slice, fc(·) is a fully connected layer, tanh(·) is a hyperbolic tangent activation function, and σ(·) is a sigmoid activation function.

After acquiring the weights, the global graph feature gᵢ of each time slice of one sample is weighted and summed to obtain the weighted global graph feature gᵢ^{attn}, which are specifically expressed as follows:

$${g}_{i}^{attn} = {g}_{i} ⊙ {exp\_as}_{gi} ( {score}_{i} )$$

where exp_as_{g}_{ᵢ}(·) is a function, which is used to expand the dimension of the parameters to the same as gᵢ; ⊙ indicates that the global graph feature gᵢ is multiplied by the corresponding element of scoreᵢ after the dimension is expanded.
Step 9: the feature is classified. An adaptive average pooling function is used to aggregate the weighted global graph feature gᵢ^{attn}, and then the aggregated feature is classified by using a linear layer and a Softmax activation function to obtain the prediction result y, which are specifically expressed as follows:

$$y = Softmax ( W \cdot AdaptiveAvgPool ( {g}_{i}^{attn} ) + b )$$

where W is a weight matrix of the linear layer, and b is a bias vector of the linear layer. AdaptiveAvgPool is adaptive average pooling.

It is judged whether the predictor has observed the target object according to the obtained prediction result y.

In this embodiment, the trained loss function uses the cross entropy loss function.

After the training is completed, the EEG data collected from the subjects is preprocessed and input to the model for classification to obtain the prediction results.

The present disclosure is compared with some brain-computer target reading methods with superior effects on a disclosed RSVP target detection data set. The data set includes 14 subjects. Each subject needs to detect the target picture including pedestrians in the picture sequence presented at a high speed. In the present disclosure, the method is tested using a leave-one-out method, that is, EEG data of a subject is selected as a test set, and the EEG data of the remaining subjects is selected as a training set. Furthermore, the performance of the method is evaluated using the Balanced Classification Accuracy (BCA). The accuracy calculation formula is as follows:

$$BCA = \frac{1}{\vert K \vert} {\sum }_{k = 1}^{\vert K \vert} \frac{{tp}_{k}}{{n}_{k}}$$

where tpₖ represents the number of correctly classified samples belonging to the category k, and nₖ represents the total number of samples belonging to the category k. The following table shows the effect of using the method of the present disclosure to classify the EEG data, and the results show the average balanced classification accuracy of 14 subjects.

| | | | | | The |
|---|---|---|---|---|---|
| | | | | | present |
| Model | EEGNet | Deep4Net | TIDNet | EEGITNet | disclosure |
| Average | 84.84% | 84.37% | 83.27% | 85.11% | 90.22% |
| accuracy | | | | | |

As can be seen from the data in the table, the method of the present disclosure is greatly improved compared with the prior art, and is improved by at least 5% compared with the prior art. Therefore, the effectiveness of the method according to the present disclosure is proved.

What is claimed is:

1. A brain-computer target reading method based on a dynamic graph representation network, comprising:
Step 1: acquiring Electroencephalography (EEG) data to obtain a data set comprising a plurality of samples;
Step 2: slicing each sample to obtain a plurality of time slices xᵢ;
Step 3: for each sample, constructing a dynamic temporal graph; wherein the dynamic temporal graph comprises a constructed sequence graph Gᵢ corresponding to each time slice xᵢ;
for each sequence graph Gᵢ, constructing an adjacency matrix Aᵢ using two one-dimensional vectors;
Step 4: extracting a time domain feature; wherein, the time domain feature \tilde{x}ₗ of each time slice xᵢ is extracted by two-dimensional convolution, and zero-padding is performed on the time slice xᵢ, such that dimensions of the time slice xᵢ and the time domain feature \tilde{x}ₗ are same;
Step 5: extracting a graph feature; wherein, the graph feature hᵢ of each sequence graph Gᵢ is extracted using a dynamic graph homogeneous network;
Step 6: purifying the graph feature hᵢ using a dual-branch graph pooling module; wherein the dual-branch graph pooling module performs a graph pooling operation based on local topology information and a graph pooling operation based on global topology information on the graph feature hᵢ respectively, and combines obtained graph features to obtain a pooled feature hᵢ^{pool};
Step 7: extracting a global graph feature gᵢ of each sequence graph based on the pooled feature hᵢ^{pool};
Step 8: extracting a task-related feature from global graph features gᵢ of all sequence graphs to obtain a weighted global graph feature gᵢ^{attn};
Step 9: using an adaptive average pooling function to aggregate the weighted global graph feature gᵢ^{attn}, and classifying a obtained feature to obtain a prediction result y; and judging whether a target object is observed by a subject when collecting an EEG signal according to the prediction result y.

2. The brain-computer target reading method based on the dynamic graph representation network according to claim 1, wherein an expression of the adjacency matrix Aᵢ is:
$$\begin{matrix}{A}_{i} = tan h ( Φ \cdot {Ψ}^{T} - Ψ \cdot {Φ}^{T} ) \\ idx , idy = arg top k ( {A}_{i} [ : , : ] ) \\ Ai [ - idx , - idy ] = 0\end{matrix}$$
where tanh(·) is a hyperbolic tangent activation function; Φ and Ψ are two learnable one-dimensional vectors; argtopk(·) is a return function for a maximum element subscript; k is a preset parameter.

3. The brain-computer target reading method based on the dynamic graph representation network according to claim 1, wherein an expression of extracting the graph feature hᵢ in Step 5 is:
$${h}_{i}^{v} = MLP ( ( 1 + ϵ ) \cdot \tilde{{x}_{ι}^{v}} + \tilde{{x}_{ι - 1}^{v}} + {\sum }_{u ∈ 𝒩 ( v )} {w}_{ij} \cdot \tilde{{x}_{ι}^{u}} )$$
where hᵢ^{v} represents a feature of a node v in a sequence graph Gᵢ after passing through the dynamic graph homogeneous network; MLP(·) is a multi-layer perceptron process; wᵢⱼ is an edge weight obtained from the adjacency matrix Aᵢ, and ∈ is a learnable parameter; \tilde{x}ₗ^{v} and \tilde{x}ₗ₋₁^{v} are time domain features corresponding to a single node in two adjacent time slices, respectively.

4. The brain-computer target reading method based on the dynamic graph representation network according to claim 1, wherein Step 6 comprises:
6-1: pooling the graph feature based on a local topology;
wherein, a channel topology graph of a sample is divided into five areas according to a frontal lobe, a parietal lobe, an occipital lobe, a left temporal lobe and a right temporal lobe; an importance score of each node in each area is calculated respectively; a plurality of nodes with highest importance in each area are selected to pool the graph feature hᵢ to obtain a locally pooled graph feature hᵢ^{local_pool},
6-2: pooling the graph feature hᵢ using a method based on global topology via an expression:
$${h}_{i}^{global\_pool} = bias ( {N}^{′} ) + {{{\sum }^{N - 1}}_{k = 0}}^{} weight ( {N}^{′} , k ) ★ {h}_{i}$$
where * represents an effective two-dimensional cross-correlation operator; N and N″ represent a number of EEG channels before and after global topology pooling, respectively, and hᵢ^{global_pool} represents a sequence graph feature after global topology pooling; and
6-3: performing fusion of results of dual-branch pooling using an expression:
$${h}_{i}^{pool} = Conv 2 d ( {h}_{i}^{global\_pool} + {h}_{i}^{local\_pool} )$$
wherein Conv2d(·) is a two-dimensional convolution operation.

5. The brain-computer target reading method based on the dynamic graph representation network according to claim 1, wherein in Step 7, the global graph feature gᵢ is extracted by a Softmax aggregation function with an expression:
$${g}_{i} = {\sum }_{v ∈ {h}_{i}^{pool}} \frac{exp ( β \cdot v )}{{\sum }_{u ∈ {h}_{i}^{pool}} exp ( β \cdot u )} \cdot v$$
wherein v and u are nodes in the pooled feature hᵢ^{pool}, and β is a learnable parameter.

6. The brain-computer target reading method based on the dynamic graph representation network according to claim 1, wherein Step 8 comprises:
calculating a weight scoreᵢ for each time slice using an expression:
$${score}_{i} = σ ( tan h ( \frac{1}{n} {\sum }_{i = 1}^{n} fc ( {g}_{i} ) ) \cdot {g}_{i}^{T} ) ,$$
where fc(·) is a fully connected layer, tanh(·) is a hyperbolic tangent activation function, and σ(·) is a sigmoid activation function; and
after the weight is acquired, weighting and summing the global graph feature gᵢ of each time slice to obtain the weighted global graph feature gᵢ^{attn}.

7. The brain-computer target reading method based on the dynamic graph representation network according to claim 1, wherein in Step 9, aggregated features are classified by using a linear layer and a Softmax activation function to obtain the prediction result y.

8. A brain-computer target reading system, comprising a dynamic temporal graph constructing module, a dual-branch graph pooling module and a dynamic temporal attention module;
wherein the dynamic temporal graph constructing module is configured to divide an Electroencephalography sample into a plurality of time slices, construct an adjacency matrix using two learnable vectors for each time slice, and extract a graph feature of each time slice using a dynamic graph homogeneous network;
the dual-branch graph pooling module is configured to perform graph pooling operation based on local topology information and graph pooling operation based on global topology information on the graph feature, respectively, and combine obtained graph features to obtain a pooled feature;
the dynamic temporal attention module is configured to extract a global graph feature of each sequence graph based on the pooled feature, and extract a feature highly related to a task using an attention mechanism to obtain a global graph feature for prediction and classification.

9. A computer device, comprising a memory, a processor and a computer program stored in the memory and operable on the processor, wherein the memory stores the computer program; and the processor executes the brain-computer target reading method according to claim 1.
